(19) United States
(12) Plant Patent Application Publication
(10) Pub. No.: US 2002/0078479 P1
(43) Pub. Date: Jun. 20, 2002

(54) Rose plant named 'Tanabamar'

(75) Inventor: Hans-Jurgen Evers, Uetersen (DE)

Correspondence Address: Russell D. Orkin, 700 Koppers Building, 436 Seventh Avenue, Pittsburgh, PA, 15219-1818, US

(73) Assignee: Rosen Tantau Mathias Tantau Nachfolger

(21) Appl. No.: 09/742,926

(22) Filed: Dec. 20, 2000

(51) Int. Cl. A01H 5/00

(52) U.S. Cl. PLT/108

(57) ABSTRACT

The new cultivar is a variety of a shrub-like rose plant having a flat bushy growth habit. The long stem flowers are large, have an interesting coloration, and a pleasant fragrance. The plant exhibits good hardiness and resistance to most diseases.

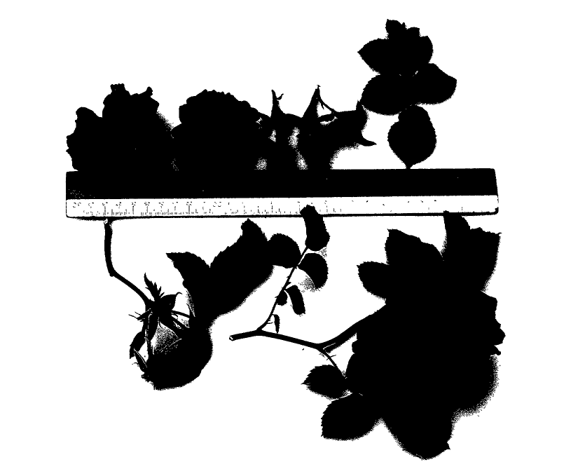

### BACKGROUND OF THE NEW PLANT

The present invention includes a new and distinct cultivar of Rosa l. known by the varietal name ‘Tanabamar’.

The new plant was discovered in 1992 by the breeder in Ueterson, Germany in a controlled breeding program. The female parent is a bi-colored F1 numbered variety from the breeder's private collection which is maintained proprietary by the assignee. The male is also a numbered variety from this collection and is a red groundcover. The plant was discovered by the breeder on the Tanotax plantation in Ueterson, Germany. The plant can be propagated asexually on Rosa canina rootstock by cuttings.

The variety is distinguishable by its large flowers, its fragrance and coloration, its shrub like appearance, and its resistance to diseases. Continued observations from the vegetative cuttings in Uetersen, Germany have confirmed that the distinguishing features of this new cultivar come true, remain stable, and are retained through successive propagations.

### DESCRIPTION OF THE DRAWING

The accompanying photographic drawing illustrates the new cultivar. Colors are described in reference to the R.H.S. Colour Chart of The Royal Horticultural Society of London.

### DESCRIPTION OF THE NEW PLANT

The following description sets forth the characteristics of the new cultivar.

The plant:

Botanical name.—Rosa l.

Size:

Height.—60 cm.

Width.—80 cm.

Habit.—Flat bushy, long stems.

Growth vigor.—Very strong.

Wood color.—Red-green.

Thorns:

Color.—Red.

Size.—Medium.

Shape.—Hooked downward.

Location on stems and branches.—Around the stem with a few beneath under the branches.

Disease resistance.—Very good.

Hardiness.—Very good.

Foliage:

Arrangement.—Compound.

Number of leaflets on typical stem leaves.—3-5.

Leaflets:

Size.—Medium to large.

Color.—Dark Green Group 131A.

Upper surface.—Green Group 131 A.

Lower surface.—Green Group 131D.

Shape.—Oval.

Texture.—Leathery.

Margin.—Medium.

Petiole rachis:

Color.—Green.

Texture.—Hardy.

Stipules:

Size.—Medium.

Shape.—Bearded.

Bud:

Size.—Small to medium.

Shape.—Rounded.

Color.—Red Group 40A.

Color changes during opening.—Strongly.

Sepals:

Color.—Green Group 139B.

Texture.—Just a few hairs.

Receptacle:

Color.—Green Group 137A.

Shape.—Funnel.

Texture.—Smooth.

Peduncle:

Color.—Green Group 141B.

Length.—Approximately 1.5 cm.

Strength.—Stiff.

Texture.—Glandular.

Flower:

Borne (number in clusters).—3-4.

Size (diameter).—Approximately 7 cm.

Form.—High-centered.

Petals:

Number per flower.—Approximately 20.

Color:

Upper surface.—Red Group 40A.

Lower surface.—Red Group 43A which changes with maturity to Red Group 43B.

Texture.—Soft

Size.—Medium; 3-4 cm.

Shape.—Spread.

Persistence.—Petals drop off cleanly before drying.

Lastingness.—Approximately 5 days.

Fragrance.—Medium.

Blooming period.—In two flushes.

Reproductive organs:

Stamens:

Number.—Medium.

Anther:

Length.—Short.

Color.—Yellow.

Filament:

Length.—Short.

Color.—Yellow-Orange.

Pollen color.—Orange.

Pistils:

Number.—Approximately 15.

Style:

Color.—Green-White.

Length.—Short.

Stigma:

Color.—Yellow-White.

Length.—Short.

I claim:

1. A new and distinct variety of rose plant substantially as shown and described.
